(19) United States
(12) Patent Application Publication
(10) Pub. No.: US 2017/0304610 A1
(43) Pub. Date: Oct. 26, 2017

(54) ADJUNCTIVE THERAPY DELIVERY USING A DBS LEAD

(71) Applicant: Boston Scientific Neuromodulation Corporation, Valencia, CA (US)

(72) Inventors: Barbara Huibregtse, Westborough, MA (US); Anne M. Pianca, Santa Monica, CA (US); Eric Schneider, Cumberland, RI (US); Lynne Swanson, Edina, MN (US)

(21) Appl. No.: 15/495,689

(22) Filed: Apr. 24, 2017

Related U.S. Application Data
(60) Provisional application No. 62/326,875, filed on Apr. 25, 2016.

(51) Int. Cl. A61N 1/05; A61N 1/36

(52) U.S. Cl. CPC A61N 1/0534; A61N 1/36082; A61N 1/36135

(57) ABSTRACT

Various aspects of the present disclosure are directed toward apparatuses, systems and methods for treating an ischemic brain injury. Certain aspects include delivering the treatment substrate to a therapy region in a patient's brain. In addition, certain aspects of the disclosure can include using a number of electrodes arranged with a deep brain stimulation lead to identify the therapy region.

### CROSS-REFERENCE TO RELATED APPLICATIONS

This application claims the benefit under 35 U.S.C. §119(e) of U.S. Provisional Patent Application Ser. No. 62/326,875, filed Apr. 25, 2016, which is incorporated herein by reference.

### TECHNICAL FIELD

The present disclosure relates to apparatuses, systems, and methods for treating an ischemic brain injury. More specifically, the disclosure relates to apparatuses, systems, and methods for delivering a treatment to a therapy region in a patient's brain.

### BACKGROUND

Neurostimulation systems may be therapeutic in a variety of diseases and disorders. For example, neurostimulation systems may be effective to treat neurological disorders, such as neurodegenerative diseases (e.g., Alzheimer's Disease, Parkinson's Disease, tremor, and epilepsy), brain ischemia, such as stroke, and limbic disorders, as well as non-neurological disorders, such as migraine headaches, obesity, and incontinence, by electrically stimulating selected portions of the brain. In a deep brain stimulation (DBS) procedure, a selected deep brain structure may be electrically stimulated.

In addition, organ injury and/or neurodegenerative diseases may cause metabolic dysfunction in organ tissue. Treating tissue regions having metabolic dysfunction may be beneficial in treatment of neurological disorders and injuries.

### SUMMARY

In Example 1, a deep brain stimulation lead includes: a plurality of electrodes configured to obtain a physiological signal indicative of a therapy region; and a lumen arranged within the deep brain stimulation lead and configured to facilitate delivery of a treatment substrate to the therapy region.

In Example 2, the lead of Example 1, further including an insulation layer arranged with the deep brain stimulation lead between the plurality of electrodes and the lumen.

In Example 3, the lead of Examples 1 or 2, wherein an interior surface of the lumen further includes a lubricious coating configured to facilitate delivery of the treatment substrate.

In Example 4, the lead of any of Examples 1-3, wherein the plurality of electrodes is arranged circumferentially around the deep brain stimulation lead.

In Example 5, the lead of any of Examples 1-4, wherein the substrate includes at least one of glucose, autologous mitochondria, pyruvate, creatine, and L-carnitine.

In Example 6, the lead of any of Examples 1-5, wherein the deep brain stimulation lead includes an external surface, and the lumen is centrally arranged within the external surface of the lead, and further including at least one port extending through the external surface to the lumen.

In Example 7, the lead of Example 6, further including at least one valve configured to close the at least one port.

In Example 8, the lead of Example 6, wherein the at least one port includes a plurality of ports arranged at a plurality of locations along the external surface of the deep brain stimulation lead, and further including a sheath configured to direct the treatment substrate through at least one of the plurality of ports to deliver the treatment substrate to the therapy region.

In Example 9, the lead of any of Examples 1-8, wherein the treatment substrate is configured to increase at least one of cellular metabolism, respiration, and function at the therapy region.

In Example 10, the lead of any of Examples 1-9, wherein the treatment substrate is configured to treat metabolic dysfunction in the therapy region.

In Example 11, the lead of any of Examples 1-10, wherein the treatment substrate is configured to treat at least one of a traumatic brain injury, ischemic stroke, Huntington's disease, Alzheimer's disease, and Amyotrophic Lateral Sclerosis (ALS).

In Example 12, the lead of any of Examples 1-11, wherein at least one of the plurality of electrodes directs stimulation energy toward the therapy region.

In Example 13, the lead of Example 12, wherein the at least one of the plurality of electrodes is arranged near the therapy region and is toggled to produce an electrical stimulation pulse, and at least another of the plurality of electrodes is toggled to withhold production of an electrical stimulation pulse.

In Example 14, the lead of Example 1, wherein the lead further includes a distal opening, and wherein the lumen is sized to receive a delivery catheter having a catheter lumen configured to deliver the treatment substrate, via one or more substrate delivery openings disposed at or near a distal end thereof, to the therapy region, the delivery catheter configured to move within the lumen such that the distal end of the delivery catheter protrudes from the distal opening of the lead, exposing the one or more substrate delivery openings to the therapy region.

In Example 15, the lead of any of Examples 1-14, wherein the lead is configured to be coupled to an implantable pulse generator.

In Example 16, a deep brain stimulation lead includes: a lead body; a plurality of electrodes arranged on the lead body, wherein at least one of the plurality of electrodes is configured to obtain a physiological signal indicative of a therapy region; and a lumen arranged within the lead body and configured to facilitate delivery of a treatment substrate to the therapy region.

In Example 17, the lead of Example 16, wherein the treatment substrate is configured to treat at least one of a traumatic brain injury, ischemic stroke, Huntington's disease, Alzheimer's disease, and Amyotrophic Lateral Sclerosis (ALS).

In Example 18, the lead of Example 16, further including an insulation layer arranged within the lead body between the plurality of electrodes and the lumen.

In Example 19, the lead of Example 16, wherein an interior surface of the lumen further includes a lubricious coating configured to facilitate delivery of the treatment substrate.

In Example 20, the lead of Example 16, wherein the treatment substrate includes at least one of glucose, autologous mitochondria, pyruvate, creatine, and L-carnitine.

In Example 21, the lead of Example 16, wherein the lead body includes an external surface, and the lumen is centrally arranged within the external surface of the lead body, and further including at least one port extending through the external surface to the lumen.

In Example 22, the lead of Example 21, wherein the at least one port includes a plurality of ports arranged at a plurality of locations along the external surface of the lead body, and further including an adjustable sheath disposed within the lumen and configured to direct the treatment substrate through at least one of the plurality of ports to deliver the treatment substrate to the therapy region.

In Example 23, the lead of Example 16, wherein the lead further includes a distal opening, and wherein the lumen is sized to receive a delivery catheter having a catheter lumen configured to deliver the treatment substrate, via one or more substrate delivery openings disposed at or near a distal end thereof, to the therapy region, the delivery catheter configured to move within the lumen such that the distal end of the delivery catheter protrudes from the distal opening of the lead, exposing the one or more substrate delivery openings to the therapy region.

In Example 24, the lead of Example 16, wherein the treatment substrate is configured to treat metabolic dysfunction in the therapy region.

In Example 25, a deep brain stimulation system includes: a lead having: a lead body; a plurality of electrodes arranged circumferentially around the lead body, wherein at least one of the plurality of electrodes is configured to obtain a physiological signal; and a lumen arranged within the lead body and configured to facilitate delivery of a treatment substrate to a therapy region; and a controller communicatively coupled to the lead, the controller configured to: receive the physiological signal; and identify the therapy region based on the physiological signal.

In Example 26, the system of Example 25, wherein at least one of the plurality of electrodes is arranged near the therapy region and is toggled to produce an electrical stimulation pulse, and at least another of the plurality of electrodes is toggled to withhold production of an electrical stimulation pulse.

In Example 27, the system of Example 25, further including a delivery catheter having a catheter lumen configured to deliver the treatment substrate, via one or more substrate delivery openings disposed at or near a distal end thereof, to the therapy region, the delivery catheter configured to move within the lumen such that the distal end of the delivery catheter protrudes from a distal opening in the lead, exposing the one or more substrate delivery openings to the therapy region.

In Example 28, the system of Example 25, the controller including an implantable pulse generator.

In Example 29, a method for treating a brain condition of a patient using a treatment substrate includes: identifying, using a lead having a plurality of electrodes arranged thereon, a therapy region based on a physiological signal obtained by at least one of the plurality of electrodes; and delivering, via a lumen arranged within the lead, the treatment substrate to the therapy region.

In Example 30, the method of Example 29, further including producing the treatment substrate, wherein producing the treatment substrate includes: isolating mitochondria from skeletal muscle of the patient; and determining that the isolated mitochondria are respiration-competent.

In Example 31, the method of Example 29, wherein delivering the treatment substrate to the therapy region includes: moving a delivery catheter within the lumen such that a distal end of the delivery catheter protrudes from a distal opening in the lead, exposing, to the therapy region, one or more substrate delivery openings disposed on the delivery catheter near a distal end of the delivery catheter.

In Example 32, the method of Example 29, further including directing electrical stimulation energy toward the therapy region using one or more of the plurality of electrodes.

In Example 33, the method of Example 29, wherein delivering the treatment substrate to the therapy region includes directing the treatment substrate through one or more of a plurality of ports extending through an external surface of the lead to the lumen.

In Example 34, the method of Example 33, wherein directing the treatment substrate through one or more of a plurality of ports includes manipulating a sheath slideably disposed within the lumen.

In Example 35, the method of Example 29, wherein the treatment substrate includes at least one of glucose, autologous mitochondria, pyruvate, creatine, and L-carnitine.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an example illustration of a deep brain stimulation (DBS) system in accordance with embodiments of the disclosure.

FIG. 2 is a plan view of a DBS system in accordance with embodiments of the disclosure.

FIG. 3 is an example cross-sectional illustration of a DBS lead in accordance with embodiments of the disclosure.

FIG. 4 is a plan view of a DBS lead implanted in tissue adjacent illustrative therapy regions in accordance with embodiments of the disclosure.

FIG. 5 is an example illustration of another DBS lead in accordance with embodiments of the disclosure.

FIG. 6 is a flow chart illustrating an example method for treating an ischemic brain condition of a patient.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various steps disclosed herein unless and except when explicitly referring to the order of individual steps.

### DETAILED DESCRIPTION

Metabolic dysfunction may occur in organ tissue as a result of a brain injury or brain disease. Using a treatment substrate (e.g., glucose, autologous mitochondria, pyruvate, creatine, L-carnitine, and/or other metabolic agents) may improve cellular metabolism, respiration and function in a subthalamic nucleus of the organ tissue having metabolic dysfunction. In certain instances, the treatment substrate may be autologous mitochondria. Respiration competent autologous mitochondria may augment or replace the function of mitochondria damaged during ischemia and allow for enhanced post-ischemic functional recovery and rescue cellular viability in a subthalamic nucleus of the organ tissue having metabolic dysfunction, which may be the result of an ischemic injury. Treatment with respiration competent mitochondria or other metabolic improving agents may facilitate correction of disorders with ischemic-like consequences due to metabolic dysfunction. In addition, the respiration competent mitochondria may allow improvement in, or a slowing of the progression of, a traumatic brain injury (such as concussion, Post-Traumatic Stress Disorder (PTSD), major depressive disorder (MDD), and etc.) Parkinson's disease, Huntington's disease, Alzheimer's disease, and/or Amyotrophic Lateral Sclerosis (ALS).

According to various embodiments, treatment of ischemic injury and/or other metabolic-related disorders may include injecting a treatment substrate in a therapy region in the brain. The therapy region may include a selected deep brain structure, e.g., the subthalamic nucleus (STN), the ventral intermedium (Vim), the internal segment of globus pallidus (GPi), the anterior thalamus, the ventrolateral thalamus (Thal), internal segment of globus pallidus (GPi), the substantia nigra pars reticulata (SNr), subthalamic nucleus (STN), the external segment of globus pallidus (GPe), and the neostriatum, is electrically stimulated. In certain instances, more than one structure may be stimulated. Additionally, treatment of ischemic injury may include injecting a treatment substrate in the therapy region in the brain and stimulation of the therapy region with a DBS lead.

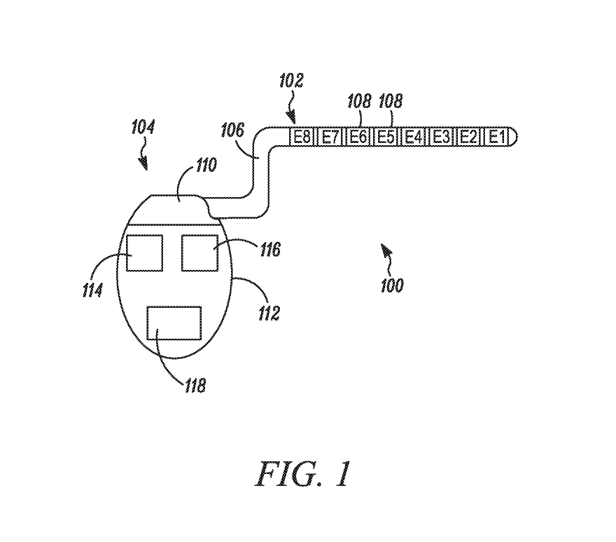

FIG. 1 is an example illustration of a deep brain stimulation (DBS) system 100, which includes a lead 102 and a controller 104 in accordance with embodiments of the disclosure. The DBS lead 102 may include an elongated cylindrical lead body 106. The DBS lead 102 includes a number of electrodes 108 arranged on the lead body 106. The electrodes 108 may be arranged circumferentially around the DBS lead 102 as ring electrodes mounted around the lead body 106. In embodiments, the electrodes 108 may extend at least approximately around the circumference of the lead body 106. In embodiments, one or more of the electrodes 108 may extend partially around the circumference of the lead body 106. In some instances, for example, the plurality of electrodes 108 may be segmented electrodes that are circumferentially and axially disposed about the lead body 106. Each of the plurality of electrodes 108 are labeled E1-E8, however the actual number and shape of leads and electrodes vary according to the application.

As shown, the DBS lead 102 is communicatively coupled to the controller 104. A connector 110 arranged with the controller 104 couples an end of the DBS lead 102 to the controller 104, thereby communicatively (e.g., electrically) coupling the electrodes 108 to the internal electronics within the controller 104. The controller 104 may also include a housing 112, which contains and houses electronic and other components. In embodiments, the controller 104 may include a pulse generator that may be implantable within a patient (e.g., an implantable pulse generator (IPG)), or configured to be positioned external to the patient. In instances where the controller 104 is implantable, the housing 112 may be formed of an electrically conductive, biocompatible material, such as titanium, and may form a hermetically sealed compartment wherein the internal electronics are protected from the body tissue and fluids.

The housing 112 may enclose sensing circuitry 114 configured to receive, from one or more of the electrodes 108, physiological signals obtained by the one or more electrodes 108. The housing 112 may also enclose pulse generation circuitry 116 that delivers stimulation energy via one or more of the electrodes 108. According to various embodiments, the sensing circuitry 114 (or aspects thereof) and/or the pulse generation circuitry 116 (or aspects thereof) may be configured to be implanted in the patient and/or disposed external to the patient. That is, for example, in embodiments, the sensing circuitry 114 and the pulse generation circuitry 116 may be integrated within a processor disposed in an implantable medical device (e.g., the controller 104) and/or an external medical device. The sensing circuitry 114 (or aspects thereof) and/or the pulse generation circuitry 116 (or aspects thereof) may be implemented in any combination of hardware, firmware, and software. For example, the sensing circuitry 114 may be, or include, a first algorithm, virtual processor, and/or process implemented by a processor, and, similarly, the pulse generation circuitry 116 circuit may be, or include, a second algorithm, virtual processor, and/or process implemented by a processor. In embodiments, the sensing circuitry 114 may be, or include, a first set of physical and/or virtual circuit elements, and, similarly, the pulse generation circuitry 116 may be, or include, a second set of physical and/or virtual circuit elements.

In embodiments, the controller 104 may include a programmable micro-controller or microprocessor, and may include one or more programmable logic devices (PLDs) or application specific integrated circuits (ASICs). In some implementations, the controller 104 may include memory as well. Although embodiments of the present system 100 are described in conjunction with a controller 104 having a microprocessor-based architecture, it will be understood that the controller 104 (or other device) may be implemented in any logic-based integrated circuit architecture, if desired. The controller 104 may include digital-to-analog (D/A) converters, analog-to-digital (A/D) converters, timers, counters, filters, switches, and/or the like.

The sensing circuitry 114 may be configured to receive a physiological signal obtained by one or more of the electrodes 108, and analyze the received physiological signal to identify a therapy region. According to embodiments, the physiological signal may include intrinsic electrical activity, a physiological response to an applied stimulation signal, and/or the like. For example, the sensing circuitry 114 may be configured to obtain a physiological signal that is a response to a stimulation signal administered using one or more of the electrodes 108, and to analyze that signal to identify a therapy location. In embodiments, the sensing circuitry 114 may be configured to evaluate motion of the patient, electrical activity of the brain, and/or other physiological signals to identify a therapy region.

The therapy region may be, in embodiments, a region including a portion of tissue (e.g., brain tissue) that is identified as being likely to be associated with a brain condition. For example, in implementations used for treating disorders affecting the motor system such as Parkinson's disease, a clinician may insert the lead 102 into a region of the patient's brain associated with the disorder, operate the controller 104 (e.g., manually, if the controller 104 is external, and via telemetry if the controller 104 is implanted), causing the controller 104 to deliver stimulation energy to a selected region via one or more of the electrodes 108. By evaluating an electrical response obtained by one or more of the electrodes 108, the controller 104 and/or the clinician may determine whether the selected region is a therapy region (e.g., the selected region may be identified as a therapy region if the physiological response to the stimulation indicates a therapeutic affect). In embodiments, the clinician may identify a therapy region by determining a region of brain tissue for which administering stimulation energy results in at least some improvement in an externally-observable symptom (e.g., jerkiness of motions, limitation of motion, etc.).

The stimulation energy may be in the form of a pulsed electrical waveform to one or more of the electrodes 108 in accordance with a set of stimulation parameters, which may be programmed into the controller 104, transmitted to the controller 104, and/or the like. Stimulation parameters may include, for example, electrode combinations that define the electrodes that are activated as anodes (positive), cathodes (negative), turned on, turned off (zero), percentage of stimulation energy assigned to each electrode (fractionalized electrode configurations), and/or electrical pulse parameters, which define the pulse amplitude (measured in milliamps or volts depending on whether the controller 104 supplies constant current or constant voltage to one or more of the electrodes 108), pulse duration (measured in microseconds), pulse rate (measured in pulses per second), and/or burst rate (measured as the stimulation on duration X and stimulation off duration Y). The pulse generation circuitry 116 may be capable of delivering the stimulation energy to the one or more of the electrodes 108 over multiple channels or over only a single channel. Stimulation energy may be used to identify therapy regions and/or to provide stimulation therapy to identified therapy regions. In embodiments, stimulation energy may be used in conjunction with treatment substrates, as described herein.

Stimulation energy may be transmitted to the tissue in a monopolar or multipolar (e.g., bipolar, tripolar, etc.) fashion. Monopolar stimulation occurs when a selected one or more of the electrodes 108 is activated and transmits stimulation energy to tissue. Bipolar stimulation, a type of multipolar stimulation, occurs when two of the electrodes 108 are activated as anode and cathode, so that stimulation energy is transmitted between the activated electrodes. Multipolar stimulation also may occur when more than two (e.g., three, four, etc.) of the electrodes 108 are activated, e.g., two as anodes and a third as a cathode, or two as cathodes and a third as an anode. In certain instances, the pulse generation circuitry 116 may individually control the magnitude of electrical current flowing through each of the electrodes. In these instances, current generators may be used to supply current-regulated amplitudes to selectively generate independent current sources for one or more of the electrodes 108.

As is further shown in FIG. 1, the DBS system 100 may include a substrate delivery system (SDS) 118 configured for facilitating the delivery of a treatment substrate, via the lead 102. The SDS 118 may be coupled to the lead 102 via a fluid conduit configured to provide a treatment substrate to a lumen arranged within the lead 102, as described in further detail below.

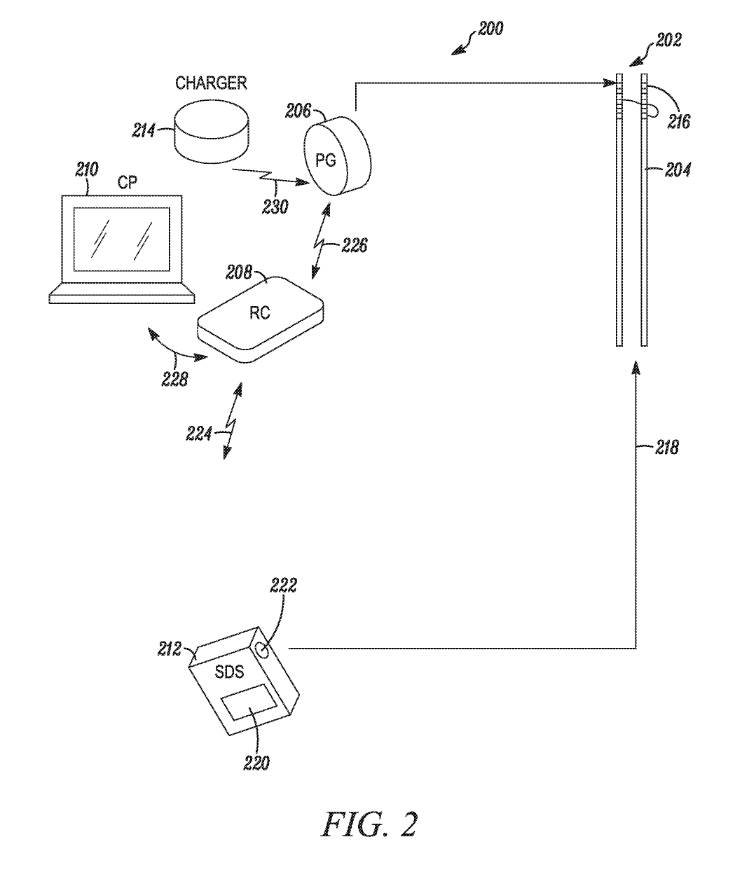

FIG. 2 is a plan view of a DBS system 200 in accordance with embodiments of the disclosure. The DBS system 200 may include at least one implantable stimulation lead 202, 204, a pulse generator (PG) 206, an external remote controller (RC) 208, a clinician's programmer (CP) 210, substrate delivery system (SDS) 212, and an external charger 214. In certain instances, the PG 206 may be electrically coupled (and, in embodiments, physically coupled) to one or both of the neurostimulation leads 202, 204, which may carry a number of electrodes 216 arranged in an array. The PG 206 may include pulse generation circuitry that delivers electrical stimulation energy in the form of a pulsed electrical waveform (i.e., a temporal series of electrical pulses) to the plurality of electrodes 216 in accordance with a set of stimulation parameters. The PG 206 may be an implantable PG (and IPG), an external PG, or may represent an operatively coupled system including one or more implantable devices and/or one or more external devices.

The SDS 212 may be coupled to the neurostimulation lead 202 via a fluid conduit (218) configured to provide a treatment substrate to a lumen arranged within the lead 202. In embodiments, the SDS 212 may include a substrate reservoir 220 configured to store a volume or other measured quantity of the treatment substrate and a metering component 222 configured to facilitate providing a specified amount of the treatment substrate from the reservoir 220 to the conduit 218. In embodiments, the metering component 222 may include a pump, a valve, a syringe, and/or the like, and may be configured to be operated electronically, manually, automatically, and/or the like. The metering component 222 may allow for sustained or controlled release of the treatment substrate after implantation. In certain embodiments, the treatment substrate may be coated on an external (abluminal) surface of one or both of the neurostimulation leads 202, 204. The treatment substrate may be coated along a length of one or both of the neurostimulation leads 202, 204, portions of the length of one or both of the neurostimulation leads 202, 204 or as a bead at a distal end of one or both of the neurostimulation leads 202, 204. Coating the neurostimulation lead 202 may allow for sustained or controlled release of the treatment substrate after implantation.

In certain instances, the DBS system 200 may use the RC 208 to telemetrically control the SDS 212 via a communications link 224. The RC 208 may be used to telemetrically control the PG 206 via a communications link 226. The RC 208 may also modify programmed stimulation parameters and/or treatment substrate delivery parameters to actively control the characteristics of the electrical stimulation energy output by the PG 206 or the delivery of the treatment substrate by the SDS 212. The RC 208 may perform these functions by indirectly communicating with the PG 206 and/or the SDS 212, through the RC 208, via a communications link 228. Alterations to the stimulation parameters, stimulation characteristics, treatment substrate delivery parameters, and/or treatment substrate delivery characteristics may be altered using the CP 210. The CP 210 may directly communicate with the PG 206 and/or the SDS 212 via a communications link (not shown). The external charger 214 may be a portable device used to charge the PG 206 via a charging link 230, which may be, e.g., an inductive charging link, a radio frequency (RF) charging link, and/or the like.

In embodiments, any one or more of the communication links 224, 226, 228, and 230 may be, or include, a wireless communication link such as, for example, a short-range radio link, such as Bluetooth, IEEE 802.11, a proprietary wireless protocol, and/or the like. In embodiments, for example, one or more of the communication links 224, 226, 228 may utilize Bluetooth Low Energy radio (Bluetooth 4.1), or a similar protocol, and may utilize an operating frequency in the range of 2.40 to 2.48 GHz. The term “communication link” may refer to an ability to communicate some type of information in at least one direction between at least two devices, and should not be understood to be limited to a direct, persistent, or otherwise limited communication channel. That is, according to embodiments, a communication link may be a persistent communication link, an intermittent communication link, an ad-hoc communication link, and/or the like. A communication link may refer to direct communications between one or more devices, and/or indirect communications that travel between the one or more devices via at least one other device (e.g., a repeater, router, hub, and/or the like). A communication link may facilitate uni-directional and/or bi-directional communication between the linked devices.

Any number of a variety of communication methods and protocols may be used, via communication links, to facilitate communication between devices in the DBS 200. For example, wired and/or wireless communications methods may be used. Wired communication methods may include, for example and without limitation, traditional copper-line communications such as DSL, broadband technologies such as ISDN and cable modems, and fiber optics, while wireless communications may include cellular communications, satellite communications, radio frequency (RF) communications, infrared communications, induction, conduction, acoustic communications, and/or the like.

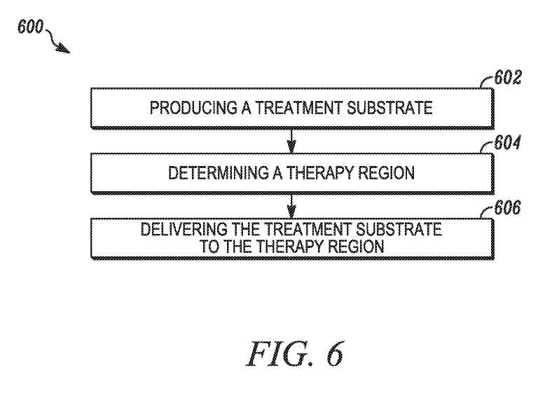

The illustrative components shown in FIGS. 1 and 2 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in any of the FIGS. 1-5 may be, in embodiments, integrated with various other components depicted therein (and/or components not illustrated), all of which are considered to be within the ambit of the disclosed subject matter. For example, the DBS leads described with reference to FIG. 1, 3, 4, or 5 may be used in the DBS system 200. Further, the DBS system 200 may be used in connection with embodiments of the method described with reference to FIG. 6.

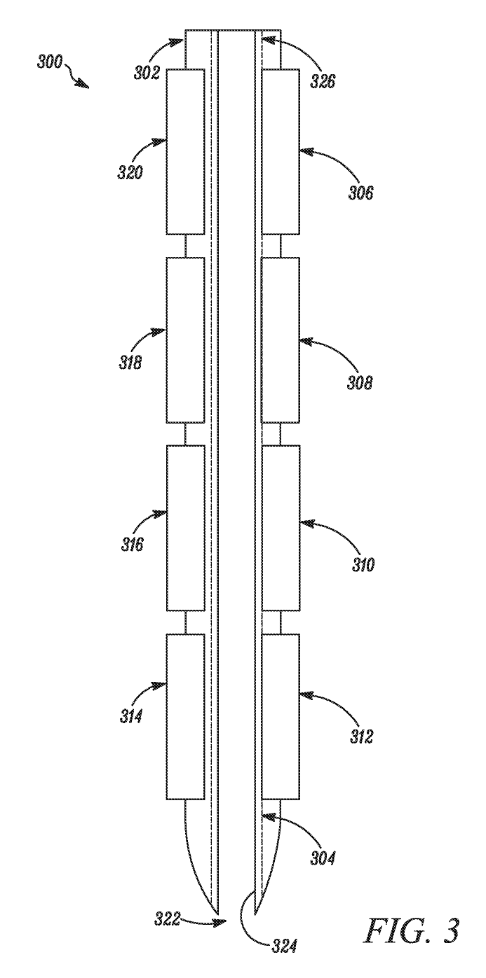

FIG. 3 is an example cross-sectional illustration of a DBS lead 300 in accordance with embodiments of the disclosure. In embodiments, the DBS lead 300 may be configured for treating an ischemic brain injury, a metabolic brain condition, and/or the like. The DBS lead 300 may include a lead body 302, a lumen 304 arranged within the lead body 302, and a number of electrodes 306, 308, 310, 312, 314, 316, 318, and 320 arranged on the lead body 302. The electrodes 306-320 may be configured to facilitate identification a therapy region based on a physiological signal obtained by one or more of the electrodes 306-320. For illustrative purposes, the electrodes 306-320 are shown extending beyond an external surface of the lead body 302, however, the electrodes 306-320 may be formed flush with the external surface of the lead body 302 (e.g., as shown in FIGS. 1, 2, 4, and 5). In embodiments, a clinician identifies the therapy region based on a change in an externally-observable symptom in response to administration of one or more electrical stimulation pulses via one or more of the electrodes 306-320. In embodiments, the one or more of the electrodes 306-320 may be configured to sense a physiological signal in response to an applied signal (e.g., a stimulation pulse). The physiological signal may be associated with an ischemic brain injury. For example, a metabolic dysfunction may occur in organ tissue as a result of a brain injury. Deep brain stimulation may increase cerebral blood flow or increase at least one of cellular metabolism, respiration, and function in a subthalamic nucleus of the organ tissue having metabolic dysfunction. Accordingly, in embodiments, the physiological signal obtained by one or more of the electrodes 306, 308, 310, 312, 314, 316, 318, 320 may be associated with an increase cerebral blood flow and/or improvement of cellular metabolism in a subthalamic nucleus of the organ tissue having metabolic dysfunction.

The lumen 304 may be configured to facilitate delivery of a treatment substrate to the therapy region. Delivery of a treatment substrate “to a therapy region” refers to delivery of a treatment substrate to a therapy region or near the therapy region. The lumen 304 may include a distal opening 322 through which the treatment substrate may be provided to the therapy region. In embodiments, the treatment substrate may be provided to the lumen 304, flow through the lumen 304, and exit the lumen 304 via the distal opening 322. In certain instances, an interior surface 324 of the lumen 304 may include a lubricious coating that is configured to facilitate delivery of the treatment substrate by promoting movement of the treatment substrate through the lumen 304 In certain instances, the lubricious coating may be polyvinyl pyrrolidone (PVP), teflon, and/or parylene. In embodiments, the DBS lead 300 may also include an insulation layer 326 arranged between the electrodes 306-320 and the lumen 304. The insulation layer 326 may be configured to prevent conduction of electricity from the electrodes 306-320 into the treatment substrate.

In embodiments, the lumen 304 may be sized to receive a delivery catheter (not shown) having a catheter lumen configured to deliver the treatment substrate, via one or more substrate delivery openings disposed at or near a distal end of the delivery catheter, to the therapy region. The delivery catheter may be configured to move within the lumen 304 such that the distal end of the delivery catheter protrudes from the distal opening 322 of the lead body 302, exposing the one or more substrate delivery openings to the therapy region.

In addition to being configured to determine a therapy region based on a physiological signal obtained by one or more of the electrodes 306-320, the DBS lead 300 may be further configured to direct stimulation toward the therapy region. Different electrode combinations of the electrodes 306-320 may be selected to change a target location of the therapy region for the stimulation. Based on analysis of the obtained physiological signal, a controller (e.g., the controller 104 depicted in FIG. 1, the PG 206 depicted in FIG. 2, the RC 208 depicted in FIG. 2, and/or the CP 210 depicted in FIG. 2) may direct a certain combination of one or more of the electrodes 306-320 to provide stimulation energy according to a certain set of stimulation parameters. In certain instances, the controller may direct an electrode combination to provide stimulation that is arranged nearest the therapy region.

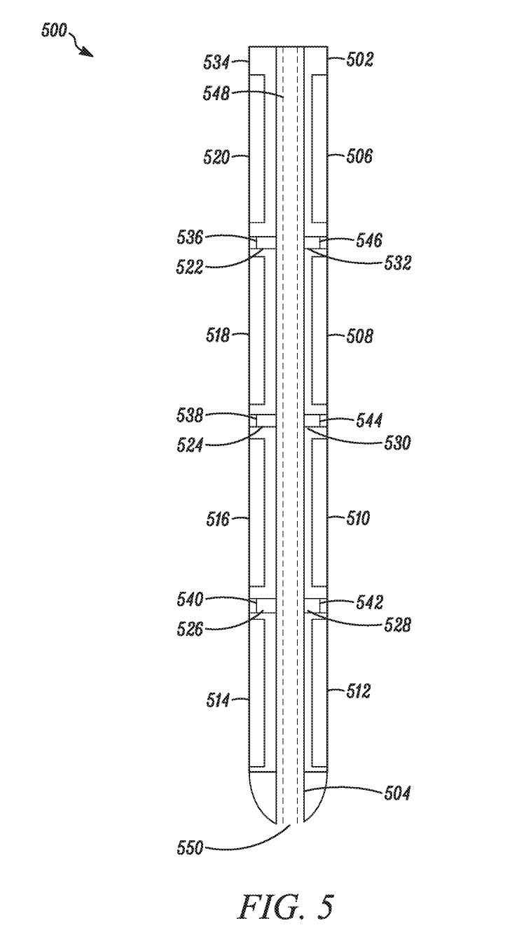

The illustrative components shown in FIG. 3 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIG. 3 may be, in embodiments, integrated with various other components depicted in FIGS. 1, 2, 4, 5, and 6 (and/or components not illustrated), all of which are considered to be within the ambit of the disclosed subject matter. For example, the DBS lead 300 may be provided as part of the DBS system 200 illustrated in and described with reference to FIG. 2. In addition, the DBS lead 300 may include a plurality or ports or a sheath as described in further detail below with reference to FIG. 5. Further, the DBS lead 300 may be used in connection with embodiments of the method described with reference to FIG. 6.

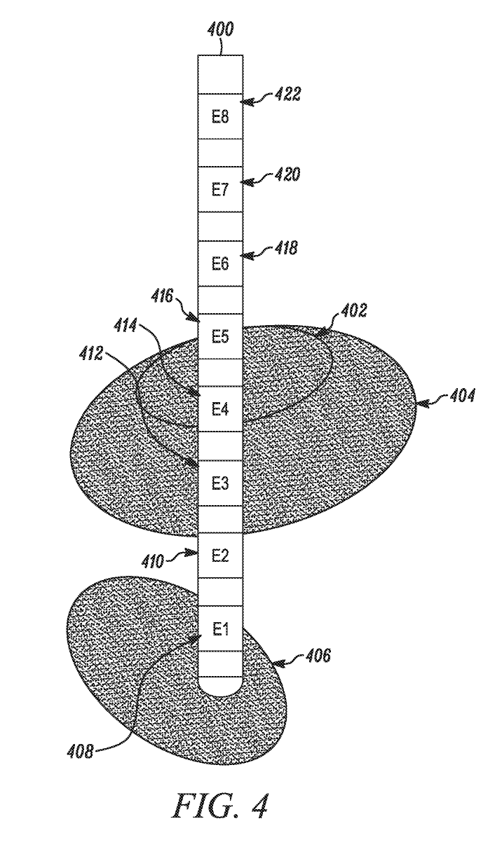

FIG. 4 is a plan view of a DBS lead 400 implanted in tissue adjacent illustrative therapy regions 402, 404, 406 in accordance with embodiments of the disclosure. The DBS lead 400 may include a plurality of electrodes 408-422. After the DBS lead 400 is implanted in tissue, a controller (e.g., the CP 210 depicted in FIG. 2) may direct certain electrode combinations to provide stimulation energy. The stimulation energy may be used for therapy and/or for identifying a therapy region by analyzing a responsive physiological signal obtained by one or more of the electrodes.

As shown in FIG. 4, the therapy regions 402, 404, 406 are located near one or more of the electrodes 408-422. For instance, a first therapy region 402 may be identified based on stimulation being supplied via two electrodes 414 and 416, and/or based on a physiological signal obtained by the two electrodes 414 and 416. Similarly, a second therapy region 404 may be identified using, for example, three electrodes 412, 414, and 416. In embodiments, the intensity level of the electrical stimulation energy conveyed via one or more of the electrodes 408-422 may also be varied. That is, for instance, the second therapy region 404 might be identified based on stimulation being supplied via the two electrodes 414 and 416 with a greater intensity level of the electrical stimulation energy than would be applied via the three electrodes 414, 416, and 418.

In embodiments, one or more of the therapy regions 402, 404, 406 may be treated at the same time. For instance, the controller may direct more than one electrode combination to provide stimulation energy. One or more of the electrodes 408-422 may be toggled to produce an electrical stimulation pulse, and at least another of the electrodes 408-422 may be toggled to withhold production of an electrical stimulation pulse. As shown with reference to the third therapy region 406, at least one electrode 422 of the electrodes 408-422 may be toggled to produce an electrical stimulation pulse, and is arranged near the therapy region 406. In this instance, the remaining electrodes 408-420 may be toggled to withhold production of an electrical stimulation pulse.

FIG. 5 is an example cross-sectional illustration of another DBS lead 500 in accordance with embodiments of the disclosure. The DBS lead 500 may be configured for treating brain condition (e.g., an ischemic brain injury) and may include a lead body 502, a lumen 504 arranged within the lead body 502, and a number of electrodes 506, 508, 510, 512, 514, 516, 518, and 520. The electrodes 506-520 may be arranged circumferentially around the lead body 502 and may be configured to obtain a physiological signal indicative of a therapy region. In embodiments, stimulation energy may be delivered by one or more of the electrodes 506-520, and one or more of the electrodes 506-520 may obtain a physiological signal in response to the stimulation energy. The physiological signal may be, for example, associated with an ischemic brain injury. In embodiments, one or more of the electrodes 506-520 may be configured to deliver electrical stimulation energy for providing therapy to adjacent tissue.

As shown in FIG. 5, the lead body 502 may include the lumen 504 centrally arranged within the lead body 502. In other embodiments, the lumen 504 may be arranged off-center within the lead body 502. The lumen 504 may be configured to facilitate delivery of a treatment substrate to the therapy region, which may be near one or more of the electrodes 506-520. In order to deliver the treatment substrate to the therapy region, the lumen 504 may include at least one port that extends through an external surface of the lead body 502 to the lumen 504. As shown in FIG. 5, the DBS lead 500 includes a number of ports 522, 524, 526, 528, 530, and 532, each of which extends through an external surface 534 to the lumen 504, thereby providing a fluid path from the lumen out of the lead 502. In embodiments, each of the ports 522-532 may be arranged and associated with one of the electrodes 506-520, though any number of other arrangements are contemplated. In certain instances, the treatment substrate may be delivered from the lumen 504 through one or more of the ports 522-532. The treatment substrate delivery may depend on the therapy region as determined using one or more of the electrodes 506-520. Additionally, or alternatively, the lumen 504 may include an opening 550 at a distal end of the lead body 502 through which the treatment substrate may be delivered.

To facilitating directing the treatment substrate to the therapy region, the DBS lead 500 may include at least one valve that is configured to close an associated port. As shown in FIG. 5, the DBS lead 500 includes a number of valves 536, 538, 540, 542, 544, and 546. Each of the valves 536-546 is associated with one of the ports 522-532 and is configured to selectively open and close the associated port. In certain instances, the valves 536-546 may be one-way valves such that the pressure differential exerted by treatment substrate through at least one of the ports 522-532 will open the one or more of the valves 536-546. In addition, the valves 536-546 may be electrostatically controlled to enable opening of a selected one or more of the valves 536-546.

In certain instances, the DBS lead 500 may include a sheath 548 that is configured to direct the treatment substrate through at least one of the ports 522-532 to deliver the treatment substrate to the therapy region. The sheath 548 may pass through the lumen 504, and may be translated along the lumen 504 to cover one or more of the ports 522-532. In addition, the sheath 548 may include associated holes or gaps that allow for covering of one or more of the ports 522-532 that are further downstream compared to others, certain combinations of ports 522-532, and/or the like. That is, for example, the sheath 548 may be configured to selectively cover port 526 and/or port 528 and not cover port 524 and/or port 530. Similarly, in another example, the sheath 548 may be configured to cover port 526 and/or port 528 and not cover port 522, port 524, port 530, and/or port 532. In embodiments, the lead 500 may include the sheath 548 as well as one or more valves 536-546, and the sheath 548 may be used in connection with the valves 536-546 to facilitate delivery of the treatment substrate to the therapy region.

The illustrative components shown in FIG. 5 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIG. 1, 2, 3, or 6 may be, in embodiments, integrated with various other components depicted therein (and/or components not illustrated), all of which are considered to be within the ambit of the disclosed subject matter. For example, the DBS lead 500 may be direct stimulation as described with reference to FIG. 4. Further, the DBS lead 500 may be provided as part of the DBS system 200 illustrated in and described with reference to FIG. 2. In addition, the DBS lead 500 may be used in connection with embodiments of the method described with reference to FIG. 6.

FIG. 6 is a flow chart illustrating an example method 600 for treating an ischemic brain condition of a patient. As is shown at block 602, embodiments of the method 600 include producing a treatment substrate. Using a treatment substrate (e.g., glucose, autologous mitochondria, pyruvate, creatine, L-carnitine, and/or other metabolic agents) as part of the method for treating an ischemic brain condition may improve cellular respiration, metabolism and function in a subthalamic nucleus of the organ tissue having metabolic dysfunction. In certain instances, the treatment substrate may include respiration competent autologous mitochondria. Treatment with respiration competent mitochondria or other metabolic improving agents may correct disorders with ischemic-like consequences due to metabolic dysfunction. The respiration competent mitochondria may allow improvement in, or slowing of the progression of, Parkinson's disease, Huntington's disease, Alzheimer's disease, and/or Amyotrophic Lateral Sclerosis (ALS). In embodiments, the step of producing the treatment substrate, shown at block 602, may include isolating mitochondria from skeletal muscle of the patient and determining that the isolated mitochondria are respiration-competent. The illustrative method described 600 may utilize one or more of the components depicted in any of FIGS. 1-5, all of which are considered to be within the ambit of the disclosed subject matter. For example, embodiments of the method 600 may include using a stylet or syringe in isolating mitochondria from skeletal muscle.

As is shown at block 604, embodiments of the method 600 for treating an ischemic brain condition of a patient also include determining a therapy region based on a physiological signal. For example, the method 600 may include using one of the DBS leads described with reference to FIGS. 1-5 to deliver stimulation energy and/or obtain a physiological signal (or signals). As is shown at block 606, embodiments of the method 600 include delivering the treatment substrate to the therapy region. Delivering the treatment substrate to the therapy region may include delivering at least one of glucose, autologous mitochondria, pyruvate, creatine, and L-carnitine to the therapy region via a lumen arranged within a DBS lead. In embodiments, delivering the treatment substrate may result in improvement in cellular metabolism, respiration and function in a subthalamic nucleus of the organ tissue having metabolic dysfunction, which may be the result of an ischemic injury.

In embodiments, delivering the treatment substrate to the therapy region includes delivering the treatment substrate through a lumen arranged within the DBS lead. The treatment substrate may be provided through the lumen directly, or using a catheter sized to be slideably disposed within the lumen. The DBS lead may include a number of ports extending through an external surface of the lead to the lumen, and delivering the treatment substrate to the therapy region may include directing the treatment substrate through one or more of the ports. In embodiments, delivering the treatment substrate to the therapy region may include directing the treatment substrate to the therapy region and directing stimulation toward the therapy region using one or more electrodes arranged circumferentially around a DBS lead.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A deep brain stimulation lead, the lead comprising:
a lead body;
a plurality of electrodes arranged on the lead body, wherein at least one of the plurality of electrodes is configured to obtain a physiological signal indicative of a therapy region; and
a lumen arranged within the lead body and configured to facilitate delivery of a treatment substrate to the therapy region.

2. The lead of claim 1, wherein the treatment substrate is configured to treat at least one of a traumatic brain injury, ischemic stroke, Huntington's disease, Alzheimer's disease, and Amyotrophic Lateral Sclerosis (ALS).

3. The lead of claim 1, further comprising an insulation layer arranged within the lead body between the plurality of electrodes and the lumen.

4. The lead of claim 1, wherein an interior surface of the lumen further comprises a lubricious coating configured to facilitate delivery of the treatment substrate.

5. The lead of claim 1, wherein the treatment substrate comprises at least one of glucose, autologous mitochondria, pyruvate, creatine, and L-carnitine.

6. The lead of claim 1, wherein the lead body comprises an external surface, and the lumen is centrally arranged within the external surface of the lead body, and further comprising at least one port extending through the external surface to the lumen.

7. The lead of claim 6, wherein the at least one port comprises a plurality of ports arranged at a plurality of locations along the external surface of the lead body, and further comprising an adjustable sheath disposed within the lumen and configured to direct the treatment substrate through at least one of the plurality of ports to deliver the treatment substrate to the therapy region.

8. The lead of claim 1, wherein the lead further comprises a distal opening, and wherein the lumen is sized to receive a delivery catheter having a catheter lumen configured to deliver the treatment substrate, via one or more substrate delivery openings disposed at or near a distal end thereof, to the therapy region, the delivery catheter configured to move within the lumen such that the distal end of the delivery catheter protrudes from the distal opening of the lead, exposing the one or more substrate delivery openings to the therapy region.

9. The lead of claim 1, wherein the treatment substrate is configured to treat metabolic dysfunction in the therapy region.

10. A deep brain stimulation system, comprising:
a lead comprising:
a lead body;
a plurality of electrodes arranged circumferentially around the lead body, wherein at least one of the plurality of electrodes is configured to obtain a physiological signal; and
a lumen arranged within the lead body and configured to facilitate delivery of a treatment substrate to a therapy region; and
a controller communicatively coupled to the lead, the controller configured to:
receive the physiological signal; and
identify the therapy region based on the physiological signal.

11. The system of claim 10, wherein at least one of the plurality of electrodes is arranged near the therapy region and is toggled to produce an electrical stimulation pulse, and at least another of the plurality of electrodes is toggled to withhold production of an electrical stimulation pulse.

12. The system of claim 10, further comprising a delivery catheter having a catheter lumen configured to deliver the treatment substrate, via one or more substrate delivery openings disposed at or near a distal end thereof, to the therapy region, the delivery catheter configured to move within the lumen such that the distal end of the delivery catheter protrudes from a distal opening in the lead, exposing the one or more substrate delivery openings to the therapy region.

13. The system of claim 10, the controller comprising an implantable pulse generator.

14. A method for treating a brain condition of a patient using a treatment substrate, the method comprising:
identifying, using a lead having a plurality of electrodes arranged thereon, a therapy region based on a physiological signal obtained by at least one of the plurality of electrodes; and
delivering, via a lumen arranged within the lead, the treatment substrate to the therapy region.

15. The method of claim 14, further comprising producing the treatment substrate, wherein producing the treatment substrate comprises:
isolating mitochondria from skeletal muscle of the patient; and
determining that the isolated mitochondria are respiration-competent.

16. The method of claim 14, wherein delivering the treatment substrate to the therapy region comprises: moving a delivery catheter within the lumen such that a distal end of the delivery catheter protrudes from a distal opening in the lead, exposing, to the therapy region, one or more substrate delivery openings disposed on the delivery catheter near a distal end of the delivery catheter.

17. The method of claim 14, further comprising directing electrical stimulation energy toward the therapy region using one or more of the plurality of electrodes.

18. The method of claim 14, wherein delivering the treatment substrate to the therapy region comprises directing the treatment substrate through one or more of a plurality of ports extending through an external surface of the lead to the lumen.

19. The method of claim 18, wherein directing the treatment substrate through one or more of a plurality of ports comprises manipulating a sheath slideably disposed within the lumen.

20. The method of claim 14, wherein the treatment substrate comprises at least one of glucose, autologous mitochondria, pyruvate, creatine, and L-carnitine.
